(19) United States
(12) Patent Application Publication
(10) Pub. No.: US 2023/0084773 A1
(43) Pub. Date: Mar. 16, 2023

(54) METHODS OF DETECTING SOMATIC MUTATIONS

(71) Applicant: The Regents of the University of California, Oakland, CA (US)

(72) Inventor: Alain Hunter Shain, San Francisco, CA (US)

(73) Assignee: The Regents of the University of California, Oakland, CA (US)

(21) Appl. No.: 17/802,084

(22) PCT Filed: Feb. 24, 2021
(86) PCT No.: PCT/US2021/019375

§ 371 Date: Aug. 24, 2022

Related U.S. Application Data
(60) Provisional application No. 62/981,435, filed on Feb. 25, 2020.

(51) Int. Cl. C12Q 1/6886

(52) U.S. Cl. CPC C12Q 1/6886; C12Q 1/6869

(57) ABSTRACT

Methods for detecting somatic mutations in single cells are described. Specifically, the disclosure provides methods of identifying somatic mutations in individual cells, comprising: providing a compartment containing only one somatic cell; generating genomic DNA and mRNA sequencing reads from the somatic cell; identifying potential mutations from the genomic DNA and mRNA sequencing reads relative to a control sequence; discarding artifact differences in the sequencing reads, thereby identifying somatic mutations in the individual cells compared to a control sequence.

### CROSS-REFERENCE TO RELATED PATENT APPLICATIONS

The present application claims benefit of priority to U.S. Provisional Patent Application No. 62/981,435, filed Feb. 25, 2020, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under grant no. K22 CA217997 awarded by the National Institutes of Health. The government has certain rights in the invention.

### BACKGROUND OF THE INVENTION

Thousands of tumors have been sequenced to date, revealing the mutations driving their growth as well as the mutational processes that operated in their past. Despite this progress, the genomic landscapes of cells from normal tissues remain poorly resolved. To borrow an analogy from Peter Campbell and colleagues, “studying fully-evolved tumors is akin to observing the similarities of great musicians, but there is no substitute to studying their development in the raw, early stages” [Martincorena I, Roshan A, Gerstung M, et al., Science, 2015; 348(6237):880-6]. Moving forward, a better understanding of the genomic landscapes of cells from normal tissues will be critical to fully understand the etiologies and origins of cancer.

Most DNA sequencing studies are performed on a bulk group of cells, yielding an average signal from the complex mixture of cells that are sampled. Bulk-cell sequencing cannot detect mutations in individual cells but can reveal subclones of cells, within the sample, that share mutations. Bulk-cell sequencing of normal blood [Jaiswal S, Fontanillas P, Flannick J, et al., N Engl J Med, 2014; 371(26):2488-98], skin [Martincorena I, Roshan A, Gerstung M, et al., Science, 2015; 348(6237):880-6], esophageal mucosa [Martincorena I, Fowler J C, Wabik A, et al., Science 2018], and colonic crypts [Lee-Six H, Olafsson S, Ellis P, et al., Nature 2019; 574(7779):532-7] has identified mutations in these tissues, including the presence of pathogenic mutations, typically associated with cancer. These studies have offered valuable glimpses into the earliest phases of carcinogenesis, justifying continued investigations into the mutational landscapes of normal tissues. However, future studies would ideally be performed at the resolution of individual cells.

Genotyping an individual cell is difficult because there is only one molecule of dsDNA corresponding to each parental allele in a diploid cell. There are primarily two strategies to circumvent this bottleneck. First, an individual cell can be sequenced after amplifying its genomic DNA in vitro [Hou Y, Song L, Zhu P, et al., Cell 2012; 148(5):873-85; Xu X, Hou Y, Yin X, et al., Cell 2012; 148(5):886-95]. Unfortunately, in vitro amplification regularly fails over large stretches of the genome, reducing the sensitivity of mutation detection, and errors are frequently incorporated during amplification, diminishing the specificity of subsequent mutation calls [Gawad C, Koh W, Quake S R, Nat Rev Genet 2016; 17(3):175-88]. Alternatively, a primary cell can be clonally expanded in tissue culture, prior to sequencing, to increase genomic starting material [Behjati S, Huch M, van Boxtel R, et al., Nature 2014; 513(7518):422-5; Blokzijl F, de Ligt J, Jager M, et al., Nature 2016; 538(7624):260-4; Kucab J E, Zou X, Morganella S, et al., Cell 2019; 177(4):821-836.e16], but only stem cells can sufficiently expand in tissue culture, limiting the scope of this strategy. Here, we combine elements of each strategy, detailed below, allowing us to overcome the deficiencies of either approach alone. We apply this workflow on skin cells, with a primary focus on melanocytes.

Melanocytes give rise to melanomas, the deadliest type of skin cancer. Melanocytes reside in the epidermis, where they are subjected to high levels of environmental damage, which can ultimately lead to their malignant transformation. Unfortunately, most of what we know regarding the etiology of melanoma is restricted to epidemiologic studies. This gap in knowledge is a major obstacle to improving prevention tactics, but here, by directly genotyping individual melanocytes from normal skin, we illuminate critical insights into the causes and origins of melanoma.

### BRIEF SUMMARY OF THE INVENTION

In some embodiments, methods and compositions for identifying somatic mutations in individual cells are provided. In some embodiments, the methods comprise: providing a compartment containing only one somatic cell; generating genomic DNA and mRNA sequencing reads from the somatic cell or from expanded cells generated by expansion of the somatic cell; identifying potential mutations from the genomic DNA and mRNA sequencing reads relative to a control sequence, wherein potential mutations are differences of a sequence read from the control sequence; discarding artifact differences in the sequencing reads, wherein the artifact differences comprise one, two, or all of the following:

a. differences that occur only in an mRNA sequencing read or only in a genomic DNA sequencing read but not both, unless the difference occurs in a sequence that undergoes chromosome (e.g., X-chromosome) inactivation or is a nonsense, splice-site, or frameshift mutation that would truncate an mRNA;

b. differences that occur relative to linked haplotype SNPs, but do not occur consistently with the haplotype SNPs, unless the difference occurs in an allelic duplication;

c. differences that do not have a normal allele frequency such that the difference frequency is statistically different from a 100% (homozygous) or 50% (heterozygous) allelic frequency,

thereby identifying somatic mutations in the individual cells compared to a control sequence.

In some embodiments, the method further comprises clonally expanding the somatic cell in the compartment to generate a plurality of expanded cells, and wherein the genomic DNA and mRNA sequencing reads are generated from the expanded cells. In some embodiments, the genomic DNA and mRNA sequencing reads are generated from the somatic cell. In some embodiments, the cells are skin cells. In some embodiments, the cells are differentiated cells. In some embodiments, the method further comprises comparing the number of real mutations to a control value representing a mutational burden of a cell, thereby determining the relative mutational age of the somatic cell.

In some embodiments, generating genomic DNA sequencing reads comprises performing multiple displacement amplification (MDA) on the genomic DNA. In some embodiments, generating genomic DNA sequencing reads comprises performing whole genome sequencing on the genomic DNA.

In some embodiments, the compartment is a droplet, microfluidic vessel or a well in a microtiter dish.

### BRIEF DESCRIPTION OF THE DRAWINGS

FIG. 1A-1F. A workflow to genotype individual skin cells. 1A. Examples of healthy skin from which we genotyped individual cells. Left panel: skin from the back of a cadaver. Right panel: skin surrounding a basal cell carcinoma. 1B. Expression profiles classify the cells that we genotyped into their respective lineages. Cells are depicted in a t-SNE plot and colored by their morphology. See FIG. 5B for further details. 1C-1D. Patterns to distinguish true mutations from amplification artifacts. 1C. Mutations in expressed genes are evident in both DNA- and RNA-sequencing data, whereas amplification artifacts are not. 1D. Germline polymorphisms, distinguished here as “A” and “B” alleles, are in linkage with somatic mutations but not amplification artifacts. 1E. Variant allele fractions from an example cell indicate how we inferred the mutational status of variants outside of the expressed and phase-able portions of the genome. Variants that were validated as mutations had variant allele fractions (VAFS) around 1 or 0.5, and variants that were invalidated had lower VAFs; however, PCR biases sometimes skewed these allele fractions. Variants that could not be directly validated or invalidated were inferred by their VAF (see methods for details). The dotted line indicates the optimal VAF cut-off to distinguish somatic mutations from amplification artifacts for this particular cell's variants (see FIG. 6B for more details). 1F. Copy number was inferred from DNA- and RNA-sequencing depth as well as from allelic imbalance—an example of a cell with a gain over chr. 5q, loss of chr. 9, and loss of chr. X is shown.

FIG. 2A-2D. The genomic landscape of individual melanocytes from physiologically healthy human skin. 2A. Top panel: Mutation burden of melanocytes from physiologically normal skin of six donors across different anatomic sites. Middle panel: Number of copy number alterations identified within each melanocyte. Lower panel: The proportion of each cell's mutations that are attributable to established mutational signatures. Signatures 7a, 7b and 7c are associated with UV-radiation induced DNA damage. Hashed bars indicate that there were too few mutations for signature analysis. 2B. Mutation burden plotted again but rank ordered by median mutation burden (line) within each site. 2C. Mutation burden of melanocytes from back skin adjacent to cancer in contrast to back skin not adjacent to cancer. The median is denoted by a grey line, and the red dotted line denotes the mutation burden of melanoma. 2D. Mutation burden of melanocytes as compared to an adjacent melanoma.

FIG. 3A-3B. Pathogenic mutations in melanocytes from normal human skin. 3A. A curated list of pathogenic mutations in melanocytes found in this study. 3B. Based on the data shown here and in conjunction with previous genetic, clinical, and histopathologic observations, we propose that melanomas can evolve via distinct trajectories, depending upon the order in which mutations occur. MAPK=Mitogen-Activated Protein-Kinase.

FIG. 4. Fields of related melanocytes identified in normal human skin. Phylogenetic trees in which each line terminus corresponds to an individual cell. Mutations that are shared between cells comprise the trunk of each tree and private mutations in each cell form the branches. Trunk and branch lengths are scaled equivalently within each tree but not across trees. The proportion of mutations that can be attributed to ultraviolet radiation is annotated in the bar charts on each tree trunk or branch. Pathogenic mutations and their location on each tree are indicated in red text. “Mel”=“Melanoma”.

FIG. 5A-5B. Establishing the ethnicity of donors and identity of cells in this study. 5A. Admixture analysis of donors included in this study alongside participants from the 1000 Genomes Project. Donors in our study were genotypically most similar to European participants from the 1000 Genomes Project. EUR—European (TSI-Toscani in Italia, IBS—Iberian Population in Spain, GBR—British in England and Scotland, CEU—Utah Residents with Northern and Western European Ancestry, FIN—Finnish in Finland), AFR—African, AMR—Latin American, SAS—South Asian, and EAS—East Asian. 5B. Differential expression analysis comparing cells that were morphologically predicted to be keratinocytes, melanocytes, or fibroblasts (see FIG. 1B for more details). The top 20 differentially expressed genes for each group are shown along with gene ontology terms with significant overlap.

FIG. 6A-6D. Detection of somatic mutations in small clones of skin cells with high specificity and sensitivity. 6A. Allelic dropout declines rapidly as a function of clone size. Each data point represents the percent of germline SNP alleles that could not be detected for a given clone as a function of the number of cells within the clone. 6B. Establishing a variant allele fraction (VAF) cut-off to infer somatic mutations within a clone. The left panel depicts the VAFs for known somatic mutations and known amplification artifacts from a single clone. The right panel depicts a ROC curve, showing the VAF at which sensitivity and specificity of somatic mutation calls would be maximized when inferring the mutational status of variants based on VAF alone. Variants that fell within expressed or phaseable portions of the genome were classified as mutations or artifacts as described (see FIG. 1C,D). The remaining variants were inferred based on a VAF cut-off, which maximized sensitivity and specificity of somatic mutation calls, as shown here. 6C-D. The specificity (panel C) and sensitivity (panel D) of inferred somatic mutations as a function of clone size. The mean specificity and sensitivity of inferred somatic mutations was respectively 98.83% and 98.60% for all clones of at least 5 cells. All trendlines correspond to a moving average.

FIG. 7A-7D. Differential expression analysis revealing genes significantly associated with mutation burden. 7A. All genes differentially expressed from DeSeq2 analyses (see methods) with an adjusted p value <0.01. The top correlative and anti-correlative genes are shown and ordered by the magnitude of their Pearson's r value. 7B-7D. Gene expression versus normalised mutation burden is shown for the top correlative and anti-correlative genes, as well as for MDM2, a gene of interest. Clones included in this analysis are from anatomic sites with greater than 3 standard deviations of mutation burdens among their cells, thus demonstrating a range of mutation burdens.

FIG. 8. Copy number landscape of melanocytes from normal human skin. Copy number was inferred, as described, and segments are depicted, here, denoting gains (red) and losses (blue) for each melanocyte (rows). Note that copy number alterations over autosomes were rare, whilst the loss of one sex chromosome is a common occurrence. All X chromosome deletions in females affect the inactive X.

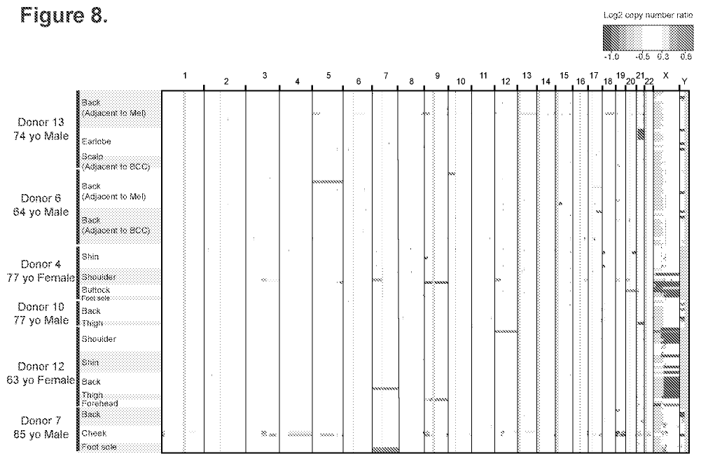

### DETAILED DESCRIPTION OF THE INVENTION

The inventor has discovered methods for efficiently identifying somatic mutations on a single-cell basis. One significant issue when starting with a small amount of genetic material is distinguishing changes that occur due to artefacts generated by DNA amplification and sequencing techniques compared to actual somatic mutations. Both artefacts and somatic mutations appear as differences when aligned to a control sequence. The inventor has discovered that several techniques can be used alone or in combination to remove changes (artefacts) that occur during amplification and sequencing that are not in the original starting nucleic acid, thereby improving precision in detection of real somatic mutations.

The method can be used for example to examine mutational burden in a sample at single-cell resolution. The number of somatic mutations in a cell reflects the mutational burden, wherein a higher mutational burden is indicated by a higher number of mutations. The number of mutations alone can be examined or the location of the mutations (e.g., in genes associated with a disease of interest, e.g., melanoma) can be targeted in an analysis. A higher mutational burden may warrant further investigation, diagnostic assays, increased occurrence of diagnostic agents, or preventative measures for a subject (e.g., a human) having a higher mutational burden.

The mutational burden is determined by comparison to one or more control sequences corresponding to (e.g., aligning with) the sequencing reads from the sample. The control sequences will generally be obtained from the same individual or same organism, and can be from a source having low, high or other levels of mutational burden. For example, an elevated the number of somatic mutations in a sample compared to a control sequence having a low or normal mutational burden can indicate the sample has an elevated mutational burden compared to the control, possibly indicating further investigation (e.g., additional diagnostic tests) was warranted. In some embodiments, the control sequences are from a different cell type of the same individual or organism as the test sample. For example, bulk blood cells can be the source of control sequences, and in some embodiments, melanocytes are the test sample.

Any cell type can be analyzed by the methods described herein. In some embodiments, the cells are differentiated cells. Exemplary cells are skin cells. In some embodiments, the cells are skin cells from a human suspected of having or at risk for melanoma. The methods described here could also be applied to measure the somatic mutations in any tissue at single-cell resolution, including tumor biopsies and peritumoral (i.e. adjacent) normal tissue

The methods described herein can generate sequencing reads from a single somatic cell (or a plurality of single cells analyzed in separate compartments in parallel). Alternatively, the single cells (e.g., in a compartment and thus isolated from other cells) can be expanded to generate additional clonal cellular material for the genetic analysis. Cellular expansion methods can be adapted depending on cell type. A variety of clonal expansion methods are known. For example, Blokzijl, et al. Nature 538:260-264 (2016); Kucab, et al. Cell 177(4):821-836, e162019; and Yoshida, et al. Nature 578:266-272(2020) describe some clonal expansion methods. An exemplary skin cell expansion method is described in the examples. The resulting expansion can in some embodiments only generate a low number of progeny cells, e.g., in some cases fewer than 10 or 100 or 1000 cells.

Compartments as used herein can be any container for holding the cell. In some embodiments, the compartment is a well, for example in a microtiter plate. In some embodiments, the compartment is a tube or other vessel. In some embodiments, the compartment is a droplet, for example such as found in water in oil emulsions, e.g., as used in digital PCR In some embodiments, the compartment is a microfluidic channel.

The methods described herein comprise generating sequencing reads from RNA (e.g., mRNA) and genomic DNA from the single somatic cell or clonally expanded cells. Any RNA or DNA sequencing methods can be used. In some embodiments, the RNA and DNA is sequences using G&Tseq (see, e.g., Macaulay I C et al., Nat Protoc 2016; 11(11):2081-103; Macaulay I C et al., Nat Methods 2015; 12(6):519-22).

As noted above, in some embodiments, specific gene sequences are targeted in the sequencing, for example by use of selective primers or by enriching for specific target sequences. Exemplary gene targets are described in but are not limited to those in the UCSF500 Cancer Gene Panel or in Shain, et al. NEJM 373:1926-1936 (2015). The number of specific target sequences can vary depending on the analysis, and in some embodiments can include 1, 2, 4, 5 or at least 10, 50, or 100 different target sequences. In other embodiments, whole genome amplification can be employed and random clones can be sequenced. In any case, sequencing reads are generated representing sequencing reads from RNA of the cell and sequencing reads from genomic DNA of the same cell.

The resulting sequencing reads can be groomed and deduplicated or otherwise cleaned. For example in some embodiments, Fastq files containing sequencing reads can undergo FastQC quality checks. The sequencing reads can initially be aligned with each other and deduplicated. Ultimately raw or groomed sequencing reads are aligned with one or more control sequence, and differences in the sequences are noted. Alignment can be performed for example on a computer.

As noted herein, differences in aligned sequences can be the result of somatic mutations in the sample cell or the differences could have been generated due to artefacts in the sequence read-generating process. Several methods can be used to remove artefact differences.

In some embodiments, genomic DNA and RNA sequencing reads for the same target sequence can be compared. Somatic mutations will be present in both RNA and DNA sequencing reads for the same target, whereas artefacts generated in the sequencing of DNA or RNA are extremely unlikely to occur at the exact same position of both RNA and DNA reads. Comparison of RNA and DNA sequences is informative if both the RNA and DNA reads would be expected to be identical. Thus, for example, differences that occur in a sequence that undergoes chromosome (e.g., X-chromosome) inactivation should not be discarded by this method because the absence of the RNA sequence could be due to inactivation. Similarly, if the difference in genomic DNA generates a nonsense, splice-site, or frameshift mutation that would truncate or alter splicing of an mRNA, those differences should not necessarily be excluded as artefacts because they would be expected to alter the RNA expression products.

In some embodiments artefact differences can be identified by comparison with haplotype SNPs. In embodiments in which more than one sequencing read is generated for a target sequence, the different reads can be compared across linked SNPs. Sequencing reads having a difference that occurs relative to linked haplotype SNPs, but do not occur consistently with the haplotype SNPs, will be an artefact and can be discarded from the listing of sequencing differences. An exception to this type of analysis occurs when the target sequence is part of an allelic (gene) duplication, meaning that there are multiple copies of similar but not identical genes in the genome. In this circumstance, for example, it can be difficult to be certain which gene allelic copy should be aligned to a RNA read.

In some embodiments, allelic frequency can be used to identify and discard artefact differences. In embodiments in which a sufficient number of sequencing reads are generated for a target sequence, an allelic frequency can be determined and those differences having a frequency that diverges from an expected frequency can be discarded. For example, alleles from a diploid organism should in general occur at 100% frequency for homozygous sequences and 50% for heterozygous sequences. If a difference has a frequency statistically different from these frequencies, it can be discarded as a likely artefact. In some embodiments one or more cut-off values are determined for particular target sequences, wherein if the allelic frequency of a difference is below or above the cut-off it is excluded. A cut-off can be deduced from the allele frequencies of known somatic mutations and known amplification artefacts, for example as determined by the methods in the two paragraphs immediately above.

As noted above, the RNA/DNA comparison, the haplotype SNP and the allelic frequency methods above can be used alone or in combination to remove artefact differences from somatic mutation differences. Thus in some embodiments, the RNA/DNA comparison and the haplotype SNP methods are both used. In some embodiments, the RNA/DNA comparison and the allelic frequency methods are both used. In some embodiments, the haplotype SNP and the allelic frequency methods are both used. In some embodiments, all three methods are used to discard differences. The remaining differences can then be considered a more precise measure of somatic mutations in the cell.

### EXAMPLE

The following examples are offered to illustrate, but not to limit the claimed invention.

### Results

### A Workflow to Genotype Individual Skin Cells

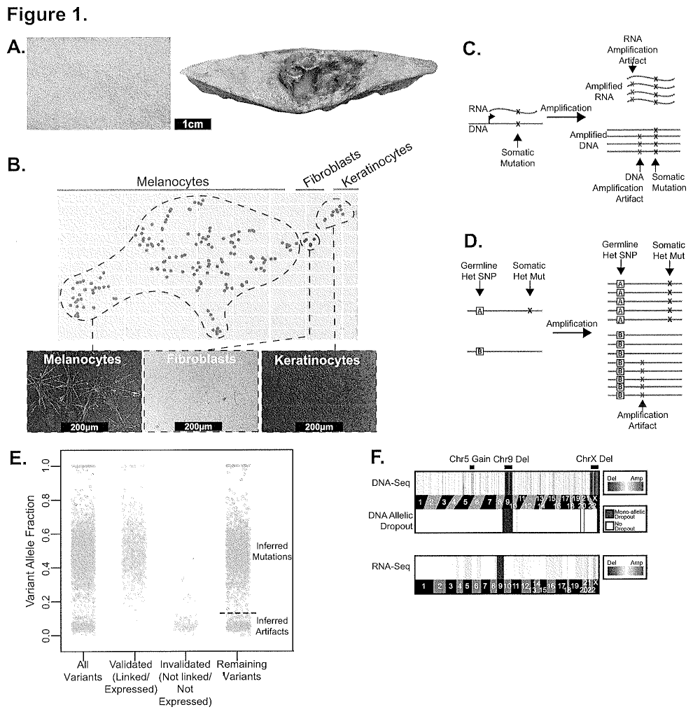

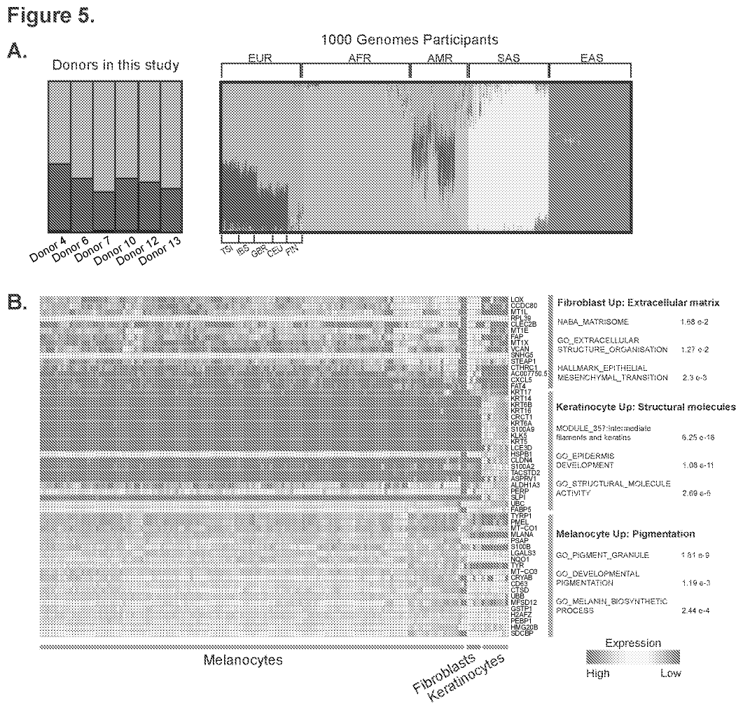

We collected physiologically normal skin, defined here as skin without palpable lesions, from 19 sites across 6 donors. Skin biopsies were obtained from cadavers with no history of skin cancer or from peritumoral tissue of donors with skin cancer (FIG. 1A). All donors were of light skin tone, European ancestry (FIG. 5A), and ranged from 63 to 85 in age.

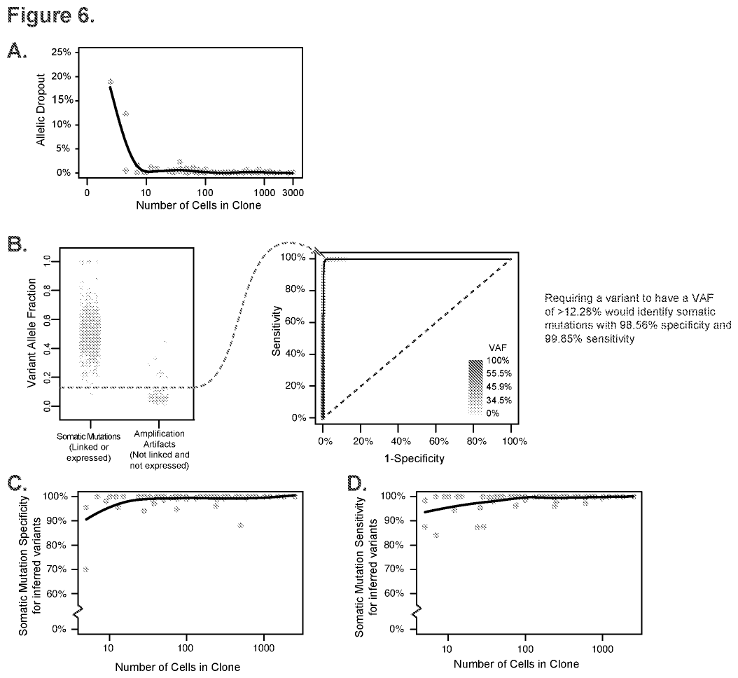

From each skin biopsy, epidermal cells were briefly established in tissue culture (1 week), subsequently single-cell sorted and clonally expanded. As expected, skin cells were unable to grow out indefinitely from a single cell, but under optimized culture conditions, melanocytes produced expansions ranging from 2-3000 cells (median 184 cells). Despite the small size of these expansions, the additional cells provided extra templates from which to genotype, which mitigated the critical bottleneck of attempting to genotype from a single cell and substantially enhanced the sensitivity of variant detection. Overall, we achieved 99.86% allelic coverage (i.e. 0.14% allelic dropout) for clones greater than 5 cells (FIG. 6A).

Next, we extracted, amplified, and sequenced both DNA and RNA from each clonal expansion, as described [Macaulay I C et al., Nat Protoc 2016; 11(11):2081-103; Macaulay I C et al., Nat Methods 2015; 12(6):519-22]. Our tissue culture conditions favored melanocyte growth, but some keratinocytes and fibroblasts also grew out. The morphology of cells and their RNA sequencing data provided confirmation of each cell's identity (FIG. 1B, 5B). Moreover, the matched DNA/RNA sequencing data improved the specificity of mutation calls because mutations in expressed genes could be cross-validated, whereas amplification artifacts could not (FIG. 1C). Finally, the matched DNA/RNA sequencing data permitted genotype/phenotype inquiries, as described in subsequent sections.

To further improve the specificity of mutation calls, we leveraged haplotype information to root out amplification artifacts. When reads are phased into their maternal and paternal haplotypes using heterozygous germline variants, neighboring somatic mutations occur within all amplified copies of that haplotype, whereas amplification artifacts rarely display this pattern (FIG. 1D) [Lodato M A, Rodin R E, Bohrson C L, et al., Science 2018; 359(6375):555-9; Bohrson C L, Barton A R, Lodato M A, et al., bioRxiv 2017; 211169. Overall, we were able to confidently distinguish true somatic mutations from amplification artifacts in the expressed and phase-able portions of the genome.

Variants that fell outside of the expressed and phase-able portions of the genome were classified as somatic mutations or artifacts based on their variant allele frequencies. Heterozygous mutations should have allele frequencies of 50%, whereas, amplification artifacts tend to have much lower allele frequencies. For each cell, we identified the variant allele frequency that would maximize the specificity and sensitivity of mutation calls, trained on the known somatic mutations and amplification artifacts from the expressed and phase-able portions of the genome (FIG. 1E, 6B-D). We applied this cut-off to those variants that fell outside of these regions to identify somatic mutations.

Finally, we deduced copy number alterations from both the DNA-seq data and the RNA-seq data using the CNVkit software suite [Talevich E et al., PLoS Comput Biol 2016; 12(4):e1004873; CNVkit-RNA: Copy number inference from RNA-Sequencing data|bioRxiv [Internet]. [cited 2019 Mar. 7]; Available from: https://www.biorxiv.org/content/10.1101/408534v1]. As an additional filter, we required that copy number alterations coincide with a concordant degree of allelic imbalance over the region affected (FIG. 1F).

In summary, we implemented a series of wet- and dry-laboratory solutions to overcome the major obstacles associated with genotyping individual cells. 133 melanocytes passed our quality control metrics and were included in all subsequent analyses.

Mutational Landscape of Melanocytes from Normal Skin

For all 133 melanocytes, we performed RNA sequencing of the entire transcriptome and DNA-sequencing on a panel of 509 genes. For a subset of 48 cells, we also performed DNA-sequencing over the entire exome. We observed an average mutation burden of 7.9 mut/Mb (mutations per megabase); however, this ranged from 0 mut/Mb to 32.3 mut/Mb, depending upon several factors.

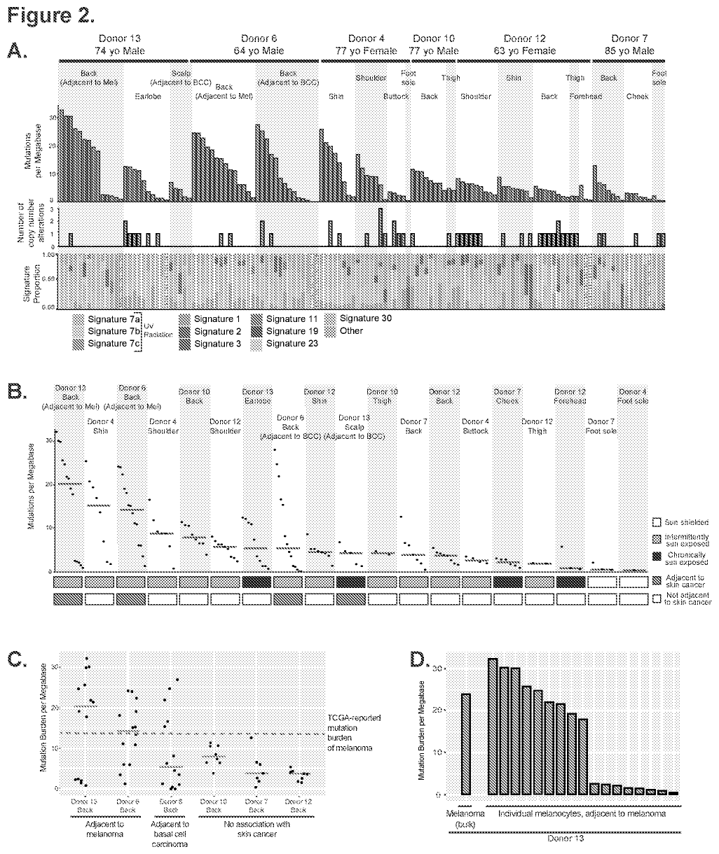

The mutation burdens of melanocytes first varied within people by anatomic site. As expected, melanocytes from sun-shielded sites had fewer mutations than those on sun-exposed sites (FIG. 2A,B). Moreover, sun-shielded melanocytes had little evidence of UV-radiation-induced mutations, whereas, this was the dominant mutational signature in melanocytes from sun-exposed skin (FIG. 2A). Within the sun-exposed group of melanocytes, cells from the back and limbs had more mutations than cells from head areas (FIG. 2A,B). Typically, skin from the back and limbs accumulates lower levels of cumulative sun exposure than skin from the head region. While our mutational observations seem incongruent with these patterns of cumulative sun exposure, our observations are consistent with the fact that melanomas are disproportionately common on intermittently sun-exposed skin as compared to other forms of skin cancer [Elwood J M, Gallagher R P, Int J Cancer 1998; 78(3):276-80; Nehal K S, Bichakjian C K, N Engl J Med 2018; 379(4):363-74].

The mutation burdens of melanocytes also varied between people. For example, we sequenced melanocytes from a common site, the back, of five donors. Among these, the melanocytes from donors 6 and 13 harbored the highest mutation burdens (FIG. 2C), which was notable because these melanocytes were adjacent to skin cancers. To put their mutation burdens in perspective, the average mutation burden of melanoma is 14.4 mutations/Mb [Hodis E, Watson I R, Kryukov G V, et al., Cell 2012; 150(2):251-63], and nearly half of the melanocytes from the backs of donors 6 and 13 exceeded this level.

In addition to the variation in mutation burdens from person-to-person or site-to-site, there was, at times, a wide range of mutation burdens from a single site within a single person. Cells from a relatively small surface area should have a similar level of exposure to UV radiation and therefore comparable mutagenic profiles. To further understand the broad range of mutation burdens, we leveraged the RNA-sequencing data from each cell clone.

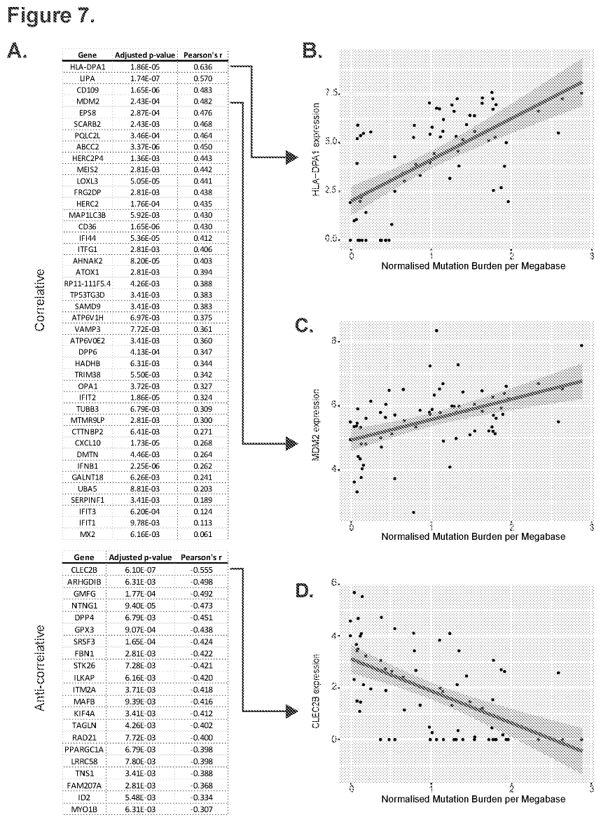

Differential expression analysis was performed to identify genes whose expression correlates with mutation burden (FIG. 7). Among the significant genes, MDM2 was more highly expressed in melanocytes with elevated mutation burdens. MDM2 protein promotes the rapid degradation of p53, raising the possibility that there is heterogeneity among melanocytes with respect to p53 activity, which could affect the ability of a cell to repair mutations or undergo DNA damage-induced cell death. Although MDM2 provides a convincing narrative to explain the mutation burden heterogeneity, it is just one out of a number of significantly correlated genes that may be taking affect. Another possibility is that melanocytes occupy different developmental states and/or cellular niches. For example, the low mutation burden melanocytes may reside, or have resided for some portion of their life, in the hair follicles rather than the interfollicular epidermis. Future studies will be needed to better resolve why melanocytes from a single site can exhibit such a broad range of mutation burdens.

Bulk melanoma tissue was available from one of the donors with melanoma, providing a rare opportunity to compare the mutation burden of a tumor to its surrounding cells. In this example, the mutation burden of the melanoma was comparable to the individual melanocytes from surrounding skin (FIG. 2D). More extensive studies are needed to validate this observation, but these findings preliminarily suggest that melanomas acquire mutations at a similar rate as surrounding skin cells, arguing against a mutator phenotype operating exclusively within melanoma cells. This would contrast with colorectal cancers, which have higher mutation burdens than surrounding normal colorectal cells [Roerink S F, Sasaki N, Lee-Six H, et al., Nature 2018; 556(7702):457-62].

Copy number alterations were relatively uncommon (FIG. 2A, middle panel), though loss of the inactive X-chromosome was recurrently detected among melanocytes in the female donors. Mosaic loss of the inactive X-chromosome has been reported in blood [Machiela M J, Zhou W, Karlins E, et al., Nat Commun 2016; 7:11843]—a finding that we extend, here, to another tissue and cell type. The rarity of autosomal copy number alterations in melanocytes from normal skin is consistent with previous reports that copy number instability is acquired during the later stages of melanoma evolution, and thus unlikely to be operative in pre-neoplastic melanocytes [Shain A H, Joseph N M, Yu R, et al., Cancer Cell 2018; 34(1):45-55.e4].

Pathogenic Mutations in Melanocytes from Normal Skin

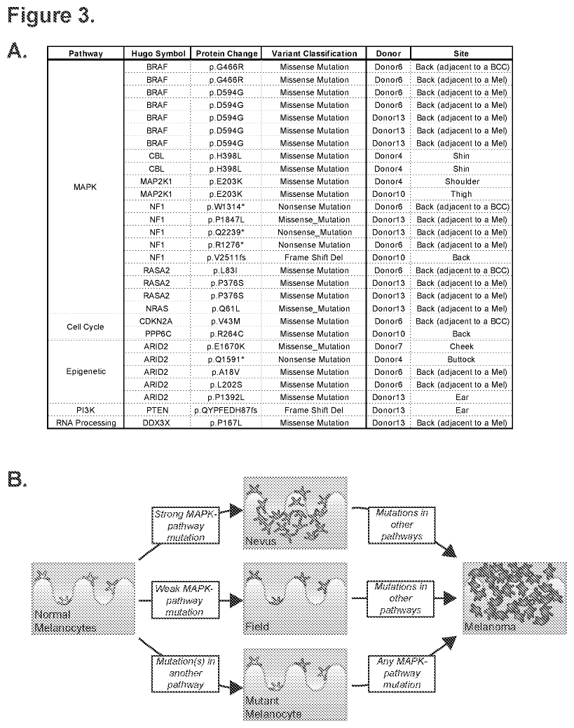

We next explored the mutations to identify those that have been previously attributed as drivers of melanoma. A set of 29 pathogenic mutations were identified, residing in 24 different cells (FIG. 3A). The mutations in melanocytes recurrently affected a core set of signaling pathways. In particular, there were numerous mutations predicted to activate the Mitogen-Activated Protein Kinase (MAPK) pathway. These include loss-of-function mutations in negative regulators of the MAPK pathway, affecting NF1, CBL, and RASA2. There were also gain- or change-of-function mutations in BRAF, NRAS, and MAP2K1, but notably lacking in our study were BRAF^{V600E} mutations—the most common mutation in the MAPK pathway occurring in melanoma [Hodis E, Watson I R, Kryukov G V, et al., Cell 2012; 150(2):251-63].

The World Health Organization (WHO) recognizes two major subtypes of cutaneous melanoma—the low cumulative sun damage (low CSD) and high cumulative sun damage (high CSD) types of melanoma. Low CSD melanomas are driven by BRAF^{V600E} mutations, whereas high CSD melanomas are driven by a more diverse collection of mutations in the MAPK pathway [Shain A H, Bastian B C, Nat Rev Cancer 2016; 16(6):345-58]. The mutations found in our study overlapped with those found in high CSD melanomas. This is notable because the precursors to high CSD melanomas are not well understood whereas low CSD melanomas are known to arise from common nevi [Shain A H, Bastian B C, Nat Rev Cancer 2016; 16(6):345-58] Shain A H, Yeh I, Kovalyshyn I, et al., N Engl J Med 2015; 373(20):1926-36]. It is likely that the BRAF^{V600E} mutation is sufficient to form a melanocytic nevus by itself, whereas the mutations, observed here, percolate in individual cells or subtle fields of cells in human skin, eventually giving rise to high CSD melanomas only after acquiring additional pathogenic mutations (FIG. 3B). This is consistent with the fact that the BRAF^{V600E} mutation is a potent activator of the MAPK pathway, while the mutations found in high CSD melanomas are thought to be weaker activators of the MAPK pathway [Yao Z, Yaeger R, Rodrik-Outmezguine V S, et al., Nature 2017; 548(7666):234-8].

We also observed driver mutations in other signaling pathways, primarily comprising mutations that disrupt chromatin remodeling factors and cell-cycle regulators. Mutations in these pathways cannot induce a melanocytic neoplasm by themselves, but they do permit the progression of precursor lesions to melanoma [Shain A H, Bastian B C., N Engl J Med 2016; 374(10):995-6]. Our findings may be clinically relevant because a subset of melanomas, known as nodular melanomas, appear suddenly and grow rapidly [Kelly J W, Chamberlain A J, et al., Aust Fam Physician 2003; 32(9):706-9]. Our data indicates that melanocytes can accumulate mutations, abrogating the secondary and tertiary barriers to tumorigenesis, without producing a neoplastic phenotype. Once these melanocytes acquire initiating mutations in the MAPK pathway, the ensuing neoplasms have the potential to skip the precursor stages and be especially aggressive (FIG. 3B).

Pathogenic mutations were enriched in melanocytes from heavily sun-damaged skin, particularly the peritumoral biopsies. We sequenced the melanoma from donor 13, allowing us to rule out the possibility that melanocytes from surrounding skin were merely an extension of the melanoma outside of the surgical margins. It seems, instead, that as skin collects sun damage, many oncogenic clones arise, enhancing the probability that one will eventually transform into melanoma.

Melanocytes can Persist as Fields of Related Cells within the Skin

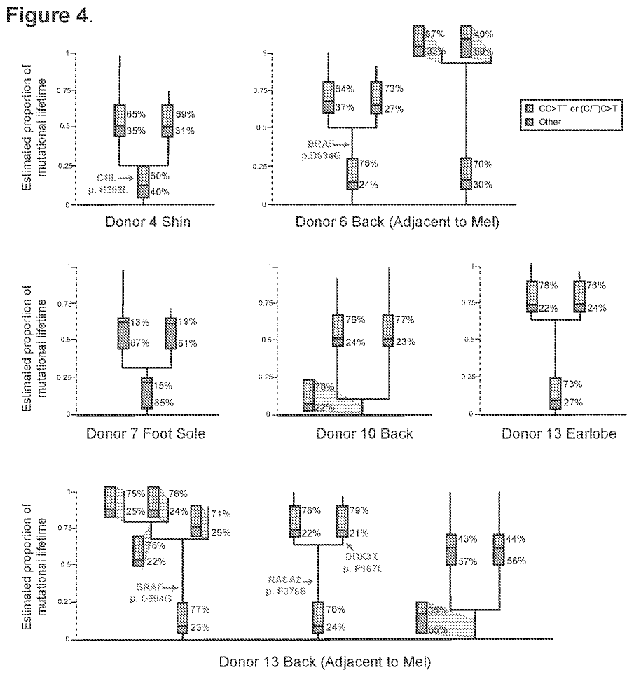

We found shared mutations between 9 separate sets of melanocytes, suggesting that these cells are related, deriving from clonal fields of melanocytes that were present in the skin (FIG. 4). Another possible explanation would be that these melanocytes are dominant clones arising during our brief period of tissue culture. To rule out this possibility, we grew normal human melanocytes from human foreskin for several months and measured their mutation burdens over time (see methods for a full description). The number of private mutations in the related sets of melanocytes, shown in FIG. 4, was many orders of magnitude higher than what would be expected from one week in tissue culture. Moreover, the private mutations from sun-exposed melanocytes showed evidence of UV-radiation-induced DNA damage (FIG. 4)—a mutational process that does not operate in tissue culture [Petljak M, Alexandrov L B, Brammeld J S, et al., Cell 2019; 176(6):1282-1294.e20].

Four of the sets of related melanocytes harbored a pathogenic mutation in the trunk of their phylogenetic trees, implicating the mutation in the establishment of the field. It is possible that the remaining fields of melanocytes had a pathogenic mutation that we did not detect or appreciate, but we favor the explanation that fields of related melanocytes can also form naturally over time, for instance, as the body surface expands.

### DISCUSSION

Here, we report the first catalogue of somatic mutations in melanocytes from human skin. There is a complex set of risk factors associated with melanoma, including cumulative levels of sun exposure as well as peak doses and timings of exposures throughout life [Schadendorf D, Akkooi A C J van, Berking C, et al., The Lancet 2018; 392(10151):971-84]. Moreover, host factors such as skin complexion, tanning ability, and DNA repair capacity influence melanoma risk [Schadendorf D, Akkooi A C J van, Berking C, et al., The Lancet 2018; 392(10151):971-84]. It is nearly impossible to quantify and integrate the effects of each of one of these variables, but we demonstrate, here, that it is feasible to directly measure the mutational damage in individual melanocytes, thus revealing the genomic consequences of these risk factors at a given time point in a person's life. We further demonstrate that heavily sun-damaged skin cells have high mutation burdens and frequently contain pathogenic mutations—these genetic features warrant further exploration as biomarkers to measure melanoma risk.

Our study also offers important insights into the origins of melanoma. Idealized progression models typically depict melanomas as passing through a series of precursor stages, but in reality, most melanomas appear suddenly, without an association to a precursor lesion [Shitara D, Nascimento M M, Puig S, et al., Am J Clin Pathol 2014; 142(4):485-91]. We show that human skin is infiltrated with individual melanocytes or subtle fields of related melanocytes harboring pathogenic mutations known to drive melanoma. These likely give rise to melanomas that appear de novo, once additional mutations have accumulated.

Finally, our genomic studies are an important resource to further understand basic melanocyte biology. For example, we found that melanocytes from sun-damaged skin have a broad range of mutation burdens, indicating that there may be distinct populations of melanocytes in human skin. Melanocytes with few mutations are likely to be more efficient at DNA repair and/or occupy privileged niches, protected from the sun. In support of the latter hypothesis, patients with vitiligo, a disease in which the immune system destroys interfollicular melanocytes, are sometimes treated with UV radiation, causing hair follicle melanocytes to migrate and colonize the interfollicular epidermis and re-pigment the area. A similar process may be operative in the general population to replenish sun-damaged melanocytes. More broadly, the genetic observations, described here, offer critical pieces of information needed to understand the homeostatic mechanisms operating on melanocytes from normal human skin.

### Methods

### Skin Tissue Collection

Physiologically normal skin tissue was collected from cadavers (up to 8 days post-mortem) or from surgical discard tissue of living donors. Skin tissue from cadavers was collected from either the UCSF Autopsy program or the UCSF Willed Body Program. Family members consented to donate tissue from the UCSF Autopsy program, and Willed-Body donors consented to donate their tissues for scientific research prior to their expiration. Surgical discard tissue was collected from donors undergoing dermatologic surgery at UCSF, and their consent was obtained at the time of surgery. Donors from the UCSF Willed Body Program have consented to have any data derived from the donation to be deidentified, stored and shared securely, and used for research as required by the Federal Privacy Act of 1974, California Information Practices Act of 1977, and HIPAA (Health Insurance Portability and Accountability Act). Donors from Dr. Sarah Arron's clinical practice have consented to the release and sharing of deidentified clinical data and genetic testing information via HIPAA as guided by the NIH National Human Genome Research Institute.

Here, we define physiologically normal skin as skin lacking palpable lesions. High resolution photos of each skin sample are available in the supplemental dataset. Skin tissue was stored at 4° C. and processed under 24 h at time of collection.

### Establishment of Epidermal Skin Cells in Tissue Culture

Skin tissue was briefly sterilized with 70% ethanol and rinsed with Hank's Balanced Salt Solution (Thermo #14175095). Excess dermis was trimmed off and skin was cut into pieces (approximately 2×2 mm²) using surgical scalpel blades. Tissue was incubated in 10 mg/ml dispase II (Thermo #17105-041) for 18 hr at 4° C. The epidermis was peeled away from the dermis, minced, incubated in 0.5% trypsin-EDTA (Thermo #15400-054) at 37° C. for 4 min, and neutralized with 0.5 mg/ml soybean trypsin inhibitor (Thermo #17075-029). Epidermal cells were plated in Medium 254 (Thermo #M254500) supplemented with human melanocyte growth supplement-2 (HGMS-2, Thermo #S0165) and antibiotic-antimycotic (Thermo #15240062). Cells were incubated at 37° C., 5% CO₂ for 7-14 days.

### CRISPR Engineering of a Subset of Cells

Initially, we presumed that it would be impossible to clonally expand single-cell sorted melanocytes from adult human skin, so we engineered mutations into the CDKN2A locus, as described [Zeng H, Jorapur A, Shain A H, et al., Cancer Cell 2018; 34(1):56-68.e9]. This decision was based on our previous success in engineering CDKN2A mutations into foreskin melanocytes and our ability to clonally expand these melanocytes, thereby producing isogenetic population of engineered melanocytes [Zeng H, Jorapur A, Shain A H, et al., Cancer Cell 2018; 34(1):56-68.e9]. However, during the course of these experiments, we recognized that control melanocytes, which were not engineered, could clonally expand under optimized media conditions, so we subsequently stopped engineering melanocytes. In total, 5 melanocytes were engineered prior to genotyping. Removal of these cell does not affect any of the conclusions from this study.

### Flow Cytometry and Cell Culture of Individual Cell Clones

Establishing epidermal cells in tissue culture produced a heterogeneous mixture of cells, comprised primarily of melanocytes and keratinocytes with some fibroblasts present. Differential trypsinization was used to separate melanocytes from keratinocytes using 0.05% trypsin-EDTA (Thermo #25300054) at 37° C. for 2 min and 10 min, respectively. Trypsin was neutralized with 0.5 mg/ml soybean trypsin inhibitor. Cells were centrifuged at 300 rpm for 5 min, resuspended in 300 μl sorting buffer (1×PBS without Ca²⁺ and Mg²⁺ (Caisson Labs #PBL-01), 1 mM EDTA (Thermo #AM9262), 25 mM HEPES, pH 7.0 (Thermo #15630130), and 1% bovine serum albumin (Thermo #BP67110)), strained using test tube with 35 μm cell strainer snap cap (Corning #352235), and single cell sorted into 96-well plates filled with 100 μl complete Medium 254 using a Sony SH800S Cell Sorter. Cell sorting was performed using a 100 μm microfluidic sorting chip with the 488 nm excitation laser without fluorescent markers.

The next day, cells were screened to decipher their morphology and confirm that each well had only one cell. Individual melanocytes were grown in CnT-40 melanocyte medium (CELLnTEC #CnT-40) supplemented with antibiotic-antimycotic. A small number of cells had keratinocyte or fibroblast morphology. Keratinocytes were grown in 50:50 complete Medium 254 and Keratinocyte-SFM media (Thermo #17005042), and fibroblasts were grown in complete Medium 254 for 10-14 days. After 10-21 days, clone sizes ranged from 2-3000 cells and ceased any further expansion, prompting us to harvest these clones at their peak cell count.

Extraction and Amplification of DNA and RNA from Each Clone

Clones of 2-3000 cells do not yield enough genomic material to directly sequence using conventional library preparation technologies. For this reason, we elected to isolate both DNA and RNA from each clone and pre-amplify the nucleic acids prior to sequencing. To do this, we utilized the G&T-Seq protocol [Macaulay I C et al., Nat Protoc 2016; 11(11):2081-103; Macaulay I C et al., Nat Methods 2015; 12(6):519-22].

G&T-Seq was performed, as described [Macaulay I C et al., Nat Protoc 2016; 11(11):2081-103; Macaulay I C et al., Nat Methods 2015; 12(6):519-22]. In brief, clones of cells were lysed in 7.5 μl RLT Plus Buffer (Qiagen #1053393). mRNA and genomic DNA were separated using a biotinylated oligo d(T)₃₀ VN mRNA capture primer (5′-biotin-triethyleneglycol-AAGCAGTGGTATCAACGCAGAGTACT30VN-3′, where V is either A, C or G, and N is any base; IDT) conjugated to Dynabeads MyOne Streptavidin C1 (Thermo #65001). cDNA was synthesized using the Smart-Seq2 protocol using SuperScript II reverse transcriptase (Thermo #18064014) and template-switching oligo (5′-AAGCAGTGGTATCAACGCAGAGTACrGrG+G-3′, where “r” indicates a ribonucleic acid base and “+” indicates a locked nucleic acid base; Qiagen). cDNA was amplified using KAPA HiFi HotStart ReadyMix kit (Roche #KK2502) and purified in a 1:1 volumetric ratio of Agencourt AMPure XP beads (Thermo #A63880). The average yield of amplified cDNA was 305 ng. Genomic DNA was purified in a 0:0.72 volumetric ratio of Agencourt AMPure XP beads and amplified using multiple displacement amplification with the REPLI-g Single Cell Kit (Qiagen #150345) to yield an average of 815 ng amplified genomic DNA per sample.

### Library Preparation and Next-Generation Sequencing of Amplified DNA and RNA

We next prepared the amplified cDNA and amplified genomic DNA for sequencing. Library preparation was performed according to the Roche Nimblegen SeqCap EZ Library protocol. In brief, 250 ng DNA input was sheared to 200 bp using Covaris E220 in a Covaris microtube (Covaris #520077). End repair, A-tailing, adapter ligation (xGen Duel Index UMI adapters; IDT), and library amplification was performed using the KAPA HyperPrep kit (Roche #KK8504) and KAPA Pure Beads (Roche #KK8001). Library quantification was performed using the Qubit dsDNA High Sensitivity kit and quantitative PCR with the KAPA Quantification kit (Roche #KK4854) on a Roche Lightcycler 480 with the QuantStudio 5 software.

Target enrichment for next-generation sequencing was performed with the UCSF500 Cancer Gene Panel (developed by the UCSF Clinical Cancer Genomics Laboratory; Roche) or the SeqCap EZ Exome+UTR library probes (Roche #06740294001). Hybridization reaction was performed using the SeqCap EZ Hybridization and Wash Kit (Roche #05634253001). xGen Universal blocking oligos (IDT #1075474), human COT 1 DNA (Thermo #15-279-011), and custom xGen Lockdown probes targeting the telomerase reverse transcriptase (TERT) promoter (IDT) were additionally added to the hybridization reaction. After library wash and PCR amplification, the captured library was quantified by Qubit and analyzed using the High Sensitivity DNA kit on Agilent's Bioanalyzer 2500.

xGen Lockdown probe sequences targeting TERT
promoter (2X tiling)

/5Biosg/GGGCACAGACGCCCAGGACCGCGCTTCCCACGTGGCGGAGGG
ACTGGGGACCCGGGCACCCGTCCTGCCCCTTCACCTTCCAGCTCCGCCTC
CTCCGCGCGGACCCCGCCCCGTCCCGAC
/5Biosg/CCCGTCCTGCCCCTTCACCTTCCAGCTCCGCCTCCTCCGCGC
GGACCCCGCCCCGTCCCGACCCCTCCCGGGTCCCCGGCCCAGCCCCCTCC
GGGCCCTCCCAGCCCCTCCCCTTCCTTT
/5Biosg/CGACCCCTCCCGGGTCCCCGGCCCAGCCCCCTCCGGGCCCTC
CCAGCCCCTCCCCTTCCTTTCCGCGGCCCCGCCCTCTCCTCGCGGCGCGA
GTTTCAGGCAGCGCTGCGTCCTGCTGCG
/5Biosg/CTTTCCGCGGCCCCGCCCTCTCCTCGCGGCGCGAGTTTCAGG
CAGCGCTGCGTCCTGCTGCGCACGTGGGAAGCCCTGGCCCCGGCCACCCC
CGCGATGCCGCGCGCTCCCCGCTGCCGA
/5Biosg/TGCGCACGTGGGAAGCCCTGGCCCCGGCCACCCCCGCGATGC
CGCGCGCTCCCCGCTGCCGAGCCGTGCGCTCCCTGCTGCGCAGCCACTAC
CGCGAGGTGCTGCCGCTGGCCACGTTCG

Libraries were sequenced on an Illumina HiSeq 2500 or Novaseq (paired end 100 bp or 150 bp). On average, we achieved 489-fold unique coverage from targeted sequencing data, 86-fold unique coverage from exome sequencing data, and 7.75 million reads/sample from RNA-sequencing data.

### Calling a Preliminary Set of Variants

Variant call format files for each clone were generated as described [Shain A H, Joseph N M, Yu R, et al., Cancer Cell 2018; 34(1):45-55.e4; Shain A H, Bagger M M, Yu R, et al., Nat Genet 2019; 51(7):1123-30]. Briefly, Fastq files underwent quality checks using FastQC and were subsequently aligned to the hg19 reference genome using the BWA-MEM algorithm (v0.7.13). BWA-aligned bam files were further groomed and deduplicated using Genome Analysis Toolkit and Picard. For each clone, variants were called using Mutect (v3.4.46) by comparing to bulk normal cells from a distant anatomic site. At this stage, the variants were composed primarily of amplification artifacts and somatic mutations. We leveraged the matched DNA/RNA sequencing data and haplotype information, detailed in the subsequent section, to distinguish between these entities.

### Harnessing the Matched DNA/RNA Sequencing Data to Remove Amplification Artifacts

The DNA and RNA from each clone were separately amplified, and consequently, amplification artifacts were unlikely to affect the same genomic coordinates in both the DNA- and RNA-sequencing reads (FIG. 1C). In contrast, somatic mutations should always overlap, assuming there was coverage of the mutant allele in both the DNA- and the RNA-sequencing data. We applied the following criteria to determine whether this assumption could be met.

To begin, we established rates of allelic dropout in our DNA- and RNA-sequencing data. From known heterozygous SNP sites, we empirically deduced that allelic dropout rates were less than 0.15% in our DNA-sequencing data. We achieved low levels of allelic dropout because of our high sequencing coverage, relatively uniform levels of coverage, and low levels of PCR-bias during amplification. Coverage in the RNA-sequencing data was more variable due to differences in gene expression, but from known heterozygous SNP sites, we empirically deduced that 15× coverage was sufficient to sample both alleles at nearly all variant sites. There were a small number of exceptions for which this did not hold true. Truncating mutations (nonsense, splice-site, and frameshift) are prone to nonsense-mediated decay and were commonly undersampled in our RNA-sequencing data relative to the wild-type allele. Also, mutations on the X-chromosome from female donors tended to be in 100% or 0% of RNA-sequencing reads, depending on whether they resided on the active or inactive X-chromosome. Aside from these examples, allelic variation in expression was minimal, particularly for highly expressed genes, as was previously reported [Reinius B, Mold J E, Ramsköld D, et al., Nat Genet 2016; 48(11):1430-5].

Based on these observations, a variant was considered a somatic mutation if it was present in both the DNA- and the RNA-sequencing data from the same clone. Conversely, a variant was considered an amplification artifact if the following conditions were met: the variant was present in the DNA-sequencing data but not the RNA-sequencing data, and there was at least 15× coverage in the RNA-sequencing data, and the variant was not truncating or on the X-chromosome. We declined to make a call in either direction for any variant that did not fulfil these conditions.

A limitation to this approach was that some variants did not reside in genes that were expressed. Nevertheless, 11.6% of variants could be classified as either a somatic mutation or amplification artifact by cross-validating the DNA/RNA sequencing data. Harnessing haplotype information to remove amplification artifacts

We also used haplotype information to distinguish between somatic mutations and amplification artifacts. Somatic mutations occur in cis with nearby germline polymorphisms, and this pattern is preserved during amplification (FIG. 1D). By contrast, amplification artifacts do not occur in complete linkage with nearby germline polymorphisms for the reasons described below (FIG. 1D).

The germline polymorphisms operate like unique molecular bar codes, designating which amplicons descended from each parental allele. The main reason why amplification artifacts are not in complete linkage with nearby polymorphisms is because there are multiple template molecules, associated with each parental allele, from which to amplify, and each template molecule can be amplified more than once—it is unlikely that the exact same mistakes are made during each independent amplification reaction over an error-free template. For example, we sequenced clonal expansions of cells, so each cell provided one molecule of double-stranded DNA from each allele. Furthermore, both strands of DNA are subject to amplification, thereby doubling the number of template molecules relative to the starting cell number. Finally, a single strand of DNA is repeatedly amplified during multiple displacement amplification, further enhancing the number of times an error-free template is utilized during amplification. Amplification artifacts therefore reveal themselves in the sequencing data by not occurring in complete linkage with nearby polymorphisms.

There was an exception for which the pattern described above did not hold true. A copy number gain or copy-number-neutral loss-of-heterozygosity (LOH) results in two or more copies of a single parental allele. If a somatic mutation occurs after the allelic duplication, then the somatic mutation would not be in complete linkage with nearby polymorphisms. Consequently, we did not apply this methodology to root out amplification artifacts over regions of the genome for which there was an allelic duplication.

A limitation to this approach is that we used short-read sequencing technologies, so some variants were too far away from the nearest polymorphic sites to be phased. Nevertheless, 14.7% of variants could be classified as either a somatic mutation or amplification artefact, using the phasing approach.

### Inferring the Mutational Status of Variants Outside of the Expressed or Phase-Able Portions of the Genome

In total, 25.1% of variants could be classified as either a somatic mutation or amplification artefact, using either the expression or the phasing approaches, described above. The remaining variants did not reside in portions of the genome that were sufficiently expressed or close enough to germline polymorphisms to permit phasing. For these variants, we inferred their mutational status from their variant allele frequency.

The majority of somatic mutations in our study were heterozygous, and these mutations, as expected, exhibited a normal distribution of mutant allele frequencies centered at 50% (FIG. 1E, 6B). The standard deviation of mutant allele frequencies in a given clone was dictated primarily by the number of starting cells, indicating that allelic biases, introduced during amplification, were the primary drivers of “noise” in our data.

By contrast, amplification artifacts exhibited a much different distribution of allele frequencies. Most amplification artifacts occurred in later rounds of amplification, and therefore had extremely low variant allele frequencies. However, a small number of amplification artifacts occurred in relatively early rounds of amplification and were disproportionately amplified thereafter. As a result, amplification artifacts exhibited a Poisson distribution of allele frequencies with a low peak but a long tail, sometimes extending into the range of allele frequencies seen for somatic mutations (FIG. 1E, 6B). As expected, the tail of this distribution was more extreme in clones with fewer starting cells because amplification biases were more exacerbated in these clones.

Due to the distinct distributions of variant allele frequencies for somatic mutations and amplification artifacts, a variant allele frequency cutoff could distinguish the vast majority of somatic mutations from amplification artifacts. However, the sensitivity and specificity of somatic mutation calls, using this approach, varied for each clone, primarily based on the clone size for the reasons described above. We were able to precisely define the sensitivity and specificity of mutation calls, and we could optimize the VAF cutoff for each clone by studying the overlap in variant allele frequencies from known somatic mutations and known amplification artifacts.

For each sample, we had a set of known somatic mutations and known amplification artifacts, situated in the expressed and phase-able portions of the genome. We were therefore able to determine the proportion of false positives and false negatives under the assumption that all variants above a given variant allele frequency were somatic mutations. Here, a “false positive” is an amplification artifact that would have been called somatic mutations, and a “false negative” is a somatic mutation that would have been called an amplification artifact. We plotted the sensitivity and specificity of mutation calls at different variant allele frequency cutoffs for each clone, and we chose the variant allele frequency cutoff that maximized these values—this value was then applied to the variants whose mutational status was unknown—i.e. the variants outside of the expressed and phase-able portions of the genome. For clones greater than 5 cells, we could typically infer somatic mutations at greater than 98% specificity and 98% sensitivity (FIG. 6C,D).

### Copy Number Analysis

Copy number alterations were inferred from both the DNA- and the RNA-sequencing data using CNVkit [Talevich E et al., PLoS Comput Biol 2016; 12(4):e1004873; CNVkit-RNA: Copy number inference from RNA-Sequencing data I bioRxiv [Internet]. [cited 2019 Mar. 7]; Available from: https://www.biorxiv.org/content/10.1101/408534v1]. We also integrated allelic frequencies from somatic mutations and germline heterozygous SNPs.

First, we inferred copy number alterations from the DNA-sequencing data. CNVkit can be run in reference or reference-free mode. We elected to run CNVkit in reference mode, and in doing so, we created several references, encompassing panels of samples without copy number alterations that were amplified and prepared for sequencing in similar batches. This approach consistently produced the least noisy copy number profiles, as compared to reference-free mode or a universal reference. All other parameters were run on their default settings.

Second, we inferred copy number alterations from the RNA-sequencing data. Briefly, CNVkit assumes the expression of a gene correlates with its copy number status. Of course, the expression of a gene is dictated by several factors, including, but not limited, to copy number. As an input, CNVkit accepts correlation values from an independent dataset between expression and copy number. Here, we included correlation values from the melanoma TCGA project. Given this input, CNVkit downweighs genes whose expression does not correlate well with copy number.

Third, we calculated allelic imbalance over germline heterozygous germline SNPs. Copy number alterations are expected to induce imbalances over these sites. Additionally, we calculated the allelic frequencies of somatic mutations across the genome, as these, too, would be modulated by copy number alterations.

Finally, we manually reviewed the copy number and variant allele information to call copy number alterations that were supported by each approach.

### Admixture Analysis (Related to FIG. 5A)

Bulk normal cells were analyzed to identify germline variants present in each studied donor. Donor ethnicity was inferred via Admixture analysis using a Bayesian modelling approach employed by the tool STRUCTURE (v2.3.4) [Pritchard J K, Stephens M, Donnelly P., Genetics 2000; 155(2):945-59]. A set of 7662 common variants (1000 genomes population allele frequency >0.05) with a sequencing depth of greater than 10 across all donors and all 2504 samples from the 1000 genomes study [Sudmant P H, Rausch T, Gardner E J, et al., Nature 2015; 526(7571):75-81] were selected. The burn-in period and analysis period were both completed with 10,000 repetitions as per the tool recommendations to achieve accurate estimations of admixture. To select an appropriate number of populations (K), the algorithm was run using K estimations of 5 to 9. A final K value of 8 was selected to appropriately cluster populations without overfitting. The data were plotted using the STRUCTURE GUI plotting tool. Ethnicity of donors within this study was inferred by their similarity to known populations within the 1000 genomes set [Sudmant P H, Rausch T, Gardner E J, et al., Nature 2015; 526(7571):75-81].

### RNA Gene Expression Analysis (Related to FIGS. 1B and 5B)

RNA sequencing reads were aligned to the transcriptome as well as the hg19 reference genome using STAR alignment tool (v2.5.1b) [Dobin A, Davis C A, Schlesinger F, et al., Bioinformatics 2013; 29(1):15-21]. Transcripts were quantified using RNA-Seq by Expectation-Maximization (RSEM) [Li B, Dewey C N., BMC Bioinformatics 2011; 12:323] and filtered to remove those with fewer than 10 reads across all samples as recommended by DESeq2 R package documentation. A variance stabilizing transformation was applied to the data and a Barnes-Hut T-distributed stochastic neighbour embedding (t-SNE) algorithm was performed to cluster related cells on the expression of the top 500 genes using the Rtsne R package (v0.15) with a perplexity of 6 over 1000 iterations.

Differential expression analysis was completed on the quantified transcript values using DESeq2 R package [Love M I, Huber W, Anders S., Genome Biol 2014; 15(12):550] (v1.22.2). Three experimental designs were produced, selecting for differentially expressed genes that are over-expressed in fibroblasts, melanocytes, and keratinocytes independently. The data were log 2 transformed and a heatmap was generated presenting the top 20 significantly differentially over-expressed genes per cell type.

Gene set enrichment analysis was performed across the significantly differentially over-expressed genes from each cell type using the Molecular Signatures Database (v6.2) webtool. The top significantly enriched pathways were examined for their relation to the cell-type of interest.

### Mutation Burden and Signature Analysis (Related to FIG. 2)

Validated somatic mutations in each clone were used to calculate mutation burden. The number of bases covered by more than 5 reads was counted in each sample and each sample's corresponding reference. The minimum value of these two numbers was used as the footprint from which to calculate a mutation burden for each sample.

To perform mutational signature analysis, surrounding genomic contexts were applied to single nucleotide variants identified in each clone using the Biostrings hg19 human genome sequence package (BSgenome.Hsapiens.UCSC.hg19 v1.4.0). Variant contexts were used to assess the proportion of each clone's mutational landscape that could be attributed to a mutagenic process using the deconstructSigs R package (v1.8.0). A set of 48 signatures recently described by Petljak et al [Petljak M, Alexandrov L B, Brammeld J S, et al., Cell 2019; 176(6):1282-1294.e20] were analysed, with particular attention paid to the single base substitution signatures 7a, 7b, and 7c that are associated with ultraviolet light exposure.

Gene Expression Correlation with Mutation Burden (Related to FIG. 7)

RNA data was used to explore the variability in mutation burdens, often observed over a single site. Sites with greater than 3 standard deviations of mutation burdens, demonstrating the presence of both high and low mutation burden clones, were selected for analysis. Mutation burdens were normalized to the median of each anatomic site. Differential expression analysis was then performed using DESeq2 R package [Love M I, Huber W, Anders S., Genome Biol 2014; 15(12):550] (v1.22.2). Genes with expression changes significantly associated (adjusted p value <0.01) with a continuous change in mutation burden are highlighted in FIG. 7.

Estimating Mutation Acquisition Over Time in Tissue Culture (Related to “Melanocytes can Persist as Fields of Related Cells within the Skin” Section of the Results)

We established skin cells in tissue culture for 7 days prior to single-cell sorting and clonal expansion. Any mutation that arose after clonal expansion would be recognizable since it would only be present in a proportion of daughter cells, thus appearing subclonal. However, mutations that arose during the brief period of tissue culture preceding clonal expansion could be mistaken as a mutation that occurred while the cell was still situated in the skin. We therefore sought to establish the rate at which melanocytes accumulate de novo mutations in tissue culture to determine whether this was a meaningful contribution to the total mutation burden that we observed in our cells.

Towards this goal, we followed the framework recently put forth by Petljak and colleagues [Petljak M, Alexandrov L B, Brammeld J S, et al., Cell 2019; 176(6):1282-1294.e20]—in that study, the authors sequenced subclones of daughter cells from common cancer lines at different generational time points, thereby revealing the mutational processes operating during their time in tissue culture. Here, we sequenced a bulk culture of normal human melanocytes derived from human foreskin to establish the germline variants and somatic mutations in the dominant clones. We continued to culture these cells, and at time points of 50, 100, and 200 days, we single-cell sorted and clonally expanded individual cells. We genotyped each clonal expansion, following the same protocol that was applied to melanocytes in this study. From these analyses, we estimate that mutations occur at a rate of 0.016 mutations/Mb per 7 days in tissue culture. To put this in perspective, the mutation burden of melanocytes from the bottom of the foot was 0.25 mutations/Mb. Based on these findings, we conclude that the number of mutations accumulated in tissue culture was negligent as compared to the number of mutations that pre-existed in melanocytes that were profiled for this study.

We also analyzed the publicly available data from Petdjak et. al. to deduce the rate at which melanoma cell lines accumulate mutations in tissue culture. From these analyses we estimate that mutations occur at a rate of 0.027 mutations/Mb per 7 days—in line with our estimates for normal human melanocytes.

Taken together, it is not surprising that the number of mutations collected from 7 days in tissue culture is negligible as compared to the number of mutations collected from decades situated in the skin.

The above examples are provided to illustrate the invention but not to limit its scope. Other variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

1. A method of identifying somatic mutations in individual cells, the method comprising:
providing a compartment containing only one somatic cell;
generating genomic DNA and mRNA sequencing reads from the somatic cell or from expanded cells generated by expansion of the somatic cell;
identifying potential mutations from the genomic DNA and mRNA sequencing reads relative to a control sequence, wherein potential mutations are differences of a sequence read from the control sequence;
discarding artifact differences in the sequencing reads, wherein the artifact differences comprise one or more or all of the following:
a. differences that occur only in an mRNA sequencing read or only in a genomic DNA sequencing read but not both, unless the difference occurs in a sequence that undergoes chromosome inactivation or is a nonsense, splice-site, or frameshift mutation that would truncate an mRNA;
b. differences that occur relative to linked haplotype SNPs, but do not occur consistently with the haplotype SNPs, unless the difference occurs in an allelic duplication;
c. differences that do not have a normal allele frequency such that the difference frequency is statistically different from a 100% (homozygous) or 50% (heterozygous) allelic frequency,
thereby identifying somatic mutations in the individual cells compared to a control sequence.

2. The method of claim 1, further comprising clonally expanding the somatic cell in the compartment to generate a plurality of expanded cells, and wherein the genomic DNA and mRNA sequencing reads are generated from the expanded cells.

3. The method of claim 1, wherein the genomic DNA and mRNA sequencing reads are generated from the somatic cell.

4. The method of claim 1, wherein the cells are skin cells.

5. The method of claim 1, wherein the cells are differentiated cells.

6. The method of claim 1, further comprising comparing the number of real mutations to a control value representing a mutational burden of a cell, thereby determining the relative mutational age of the somatic cell.

7. The method of claim 1, wherein generating genomic DNA sequencing reads comprises performing multiple displacement amplification (MDA) on the genomic DNA.

8. The method of claim 1, wherein generating genomic DNA sequencing reads comprises performing whole genome sequencing on the genomic DNA.

9. The method of claim 1, wherein the compartment is a droplet, microfluidic vessel or a well in a microtiter dish.

10. The method of claim 1, wherein the chromosome inactivation is X-chromosome inactivation.
